(19) United States
(12) Patent Application Publication
(10) Pub. No.: US 2017/0354950 A1
(43) Pub. Date: Dec. 14, 2017

(54) SILVER OXIDE MESO CRYSTAL CONTAINING SILVER PEROXIDE AND MANUFACTURING METHOD THEREFOR

(71) Applicant: MYTECH CO., LTD., Hyogo (JP)

(72) Inventors: Yuki HASEGAWA, Hyogo (JP); Katsuyuki HASEGAWA, Hyogo (JP)

(73) Assignee: MYTECH CO., LTD., Hyogo (JP)

(21) Appl. No.: 15/182,568

(22) Filed: Jun. 14, 2016

(51) Int. Cl. B01J 20/06; G01N 33/483; C01G 5/00; B01J 20/32; H01L 31/032; G01N 21/65

(52) U.S. Cl. CPC B01J 20/06; H01L 31/032; G01N 21/658; C01G 5/00; B01J 20/3234; G01N 33/4833; C01P 2006/40; G01N 2333/435

(57) ABSTRACT

A method for preparing a meso crystal of silver oxide containing silver peroxide is provided. A quantum crystal of silver thiosulfate complex on a substrate or a particle made of copper metal or copper alloy is subjected to treating by an alkaline aqueous solution containing halogen ion to obtain a meso crystal of silver oxide containing the silver peroxide. The meso crystal of silver oxide having nanometer scale, containing a silver peroxide, the silver oxide nanocrystal being a superstructure three-dimensionally arranged in the shape of a neuron provided with properties being negatively charged in water and able to be reduced to a silver nanoparticle by a laser radiation.

### FIELD OF THE INVENTION

The present invention relates to a meso crystal made of nano particles of silver oxide containing silver peroxide, and a manufacturing method for the same.

### BACKGROUND

The nano particles of metal oxide has so far been broadly used in a variety of applications which include a photo-catalyst for decomposing water, a photo-catalyst for cleaning-up environment, a material for battery, a material for sensor and a material for solar cell. The metal oxide nano particle tends to clump together disorderly and thus the surface area decreases and the interface does not fit together, whereby a lowered photo-activation and a decreased conversion efficiency of light energy may occur. In order to solve the above problems, the metal oxide will be expected to be provided with a meso crystal superstructure which can be made by self-organization of nano particles of the metal oxide (hereinafter the meso crystal is referred to as the crystal superstructure wherein nano particles are orderly and three dimensionally arranged). A variety of synthesis processes for the meso crystal have been reported. In most cases, synthesis of the meso crystal needs too much time due to the complicated procedures. Easy synthesis method has been expected for the specific kind of meso crystal.

The silver oxides, such as silver oxide (Ag₂O), silver peroxide (AgO, Ag₂O₃) are an oxide semiconductor having a forbidden band gap of 1.1 to 1.5 eV, which is almost the same as that of the crystalline Si. The silver oxides are also materials having a large absorption coefficient. The above properties give silver oxides to be used as the light-absorbing layer for the solar cell. Moreover, powders or particles of the silver oxides are also used as the positive electrode active material for the silver oxide cell.

As a method for manufacturing the silver oxide (Ag₂O), the following methods have been already proposed. 1) a method of hydrolyzing silver salt by using alkali aqueous solution under a heated and pressurized circumstance (see for example patent document 1 as described below), 2) a method of electrochemical reaction wherein a silver solution from the anode chamber and a caustic alkali from the cathode chamber are reacted in the three-chamber electrolysis vessel, provided with a salt solution in the intermediate chamber and a silver electrode as the anode. The electrolysis vessel is also provided with an anionic exchange membrane as a membrane between the anode chamber and the intermediate chamber and with a cationic exchange membrane as a membrane between the cathode chamber and the intermediate chamber, (see for example the patent document 2).

In addition, as to the silver peroxide (AgO), it has been reported that, a film of the silver peroxide was formed on a substrate by an anodic electrolysis reaction in the aqueous solution containing an aqueous solution of silver salt such as silver acetate (see the non- patent document 1 described below).

Further, a reactive sputtering method has been known for formation of the silver oxide film.

As mentioned above, a variety methods of preparing the silver oxide, such as the silver oxide (Ag₂O) or the silver peroxide (AgO) are known A wide range of applications request the method of preparing silver oxides to be more applicable without a need of huge apparatus, such as evacuating machine or heating machine.

Patent document 1: JP H11-11944

Patent document 2: JP2001-262206

Non-Patent document 1: B. E. Breyfogle, et al., J. Electrochem. Soc., 1996, 143, 2741

### SUMMARY

The subject of the present invention is to provide a silver oxide meso crystal containing a silver peroxide as well as a manufacturing method for the same. The present inventor has been forming silver complex quantum crystal from an aqueous solution of silver metal complex by a chemical reduction on a surface of copper metal or copper alloy carriers. The quantum crystal of the silver complex can be formed into a silver oxide according to the reaction mentioned below concerning an alkali aqueous solution containing halogen, such as sodium hypochlorite solution. The resulting crystal of silver oxide is made from a crystalline supersutructure in which fine particles in nanometer size are orderly and three dimensionally arranged just like a neuron network (FIG. 3). Moreover, the crystal structure is made from an anisotropic nanostructure and contains a lot of silver peroxide (FIGS. 4 and 5), so that the functionality increases due to possibility of influence by an electrode potential of the copper or copper alloy, different from the following reaction in the aqueous solution and also influence by a nucleation catalyst as a nucleus of silver chloride. As a result, a new application of the silver oxide has been developed.

Na₂S₂O₃+4NaClO+H₂O→Na₂SO₄+H₂SO₄+4NaCl (1)

Ag⁺+NaCl→AgCl+Na⁺ (2)

Ag⁺+3NaOCl→2AgCl+NaClO₃+2Na⁺ (3)

Ag⁺+OH⁻→AgOH (4)

2Ag⁺+2OH⁻→Ag₂O+H₂O (5)

Namely, the present invention provides a silver oxide meso crystal formed on a substrate or a particle of copper or copper alloy which is characterized in that the crystal contains silver peroxide and is formed as a nucleus of a silver halide by treating a quantum crystal of silver thiosulfate complex formed on the substrate or the particle of metal copper or copper alloy with an alkali aqueous solution containing halogen ion.

According to the present invention, the meso structure can be provided with a superstructure having a peroxide on a substrate or a particle of metal copper or copper alloy by a simple method of making silver complex crystals prepared by electro-chemical agglomeration, and subsequently by alkali treatment in the presence of halogen ion, resulting in a superstructure of silver oxides by self-organization, and provided with a neuron configuration arranged three dimensionally.

The silver oxide according to the present invention contains a silver peroxide, and is formed in a meso structure, which is a superstructure made by agglomeration and self-organization and arranged three dimensionally in the neuron configuration, wherein meso pores (fine pores having a diameter of 2-50 nanometers) are provided between the particles so as to be suitable for an absorption with a negative charge in a water. Thus, the silver oxide is excellent in an absorption of protein molecules. On the other hand, since the silver oxide can be reduced into silver nanoparticles due to a laser irradiation, the silver oxide is suitable for manufacturing a substrate for Raman measurement. That is, the silver oxide meso crystal containing silver peroxide according to the present invention has a negative charge in a water and can be reduced by irradiation of laser to allow silver metal nanoparticles to be precipitated. Thus, the silver oxide meso crystal has excelled in the absorption of protein molecules having a positive charge, such as a histone linked with DNA (positive charge). Therefore, the silver oxide meso crystal is used as the protein absorption chip for determining whether stem cells are differentiated or not by the Surface-Enhanced Raman Spectrum method.

The silver oxides according to the present invention contains silver oxide (Ag₂O) and silver peroxide (AgO) and also is an oxide semiconductor having a forbidden band gap of 1.1 to 1.5 eV which is the same as that of the crystalline silicon as well as a large absorption coefficient, while an uniform thin film can be formed at a necessary site on a surface of fine particles by a simple wet process. Utilization of such properties make the silver oxides to be used as a variety of applications, for example a light-absorbing layer of solar cell. A decrease in surface area and a mismatch between interfaces due to agglomeration of metal oxide nanoparticles can be prevented, thus causes of decreasing a photo-activation or a conversion efficiency of light energy can be omitted and the efficiency can be improved

### BRIEF DESCRIPTION OF THE DRAWINGS

FIG. 1 is an illustrative diagram indicating the method for producing novel SERS substrate described in the Example 1, and the upper left substrate is made by MyTech Co., Ltd. and 2 kinds of the SEM image are shown in the right side.

FIG. 2 is a SEM image of recrystallized substrate obtained in case of alkali treating (sodium hypochlorite treating) the quantum crystal substrate in the presence of halogen ion (upper figure), and a graph indicating the results of EDS spectrum (elemental analysis) of the recrystallized substrate.

FIG. 3 indicates a result of the XPS measurement of the recrystallized substrate treated by alkali.

FIG. 4 indicates a result of the XPS measurement of the recrystallized substrate treated by etching.

### DESCRIPTION OF EMBODIMENTS

The present invention is based on an idea that if the silver thiosulfate complex quantum crystal is treated by a sodium hypochlorite aqueous solution, a neuronal nano structure (meso structure) of the silver oxide containing silver chloride is formed by the chemical reaction mentioned below. The meso crystal structure has a negative charge in the aqueous solution, and thus the meso pores structure (fine pores having a diameter of 2-50 nanometers resulting from space between the particles) tends to efficiently absorb a protein having positive charge to form a charge-transfer complex, while a part of the silver oxide or the silver peroxide on its surface can be reducted into silver metal due to a laser irradiation, and as a result a surface plasmon enhancing effect occurs by the laser irradiation.

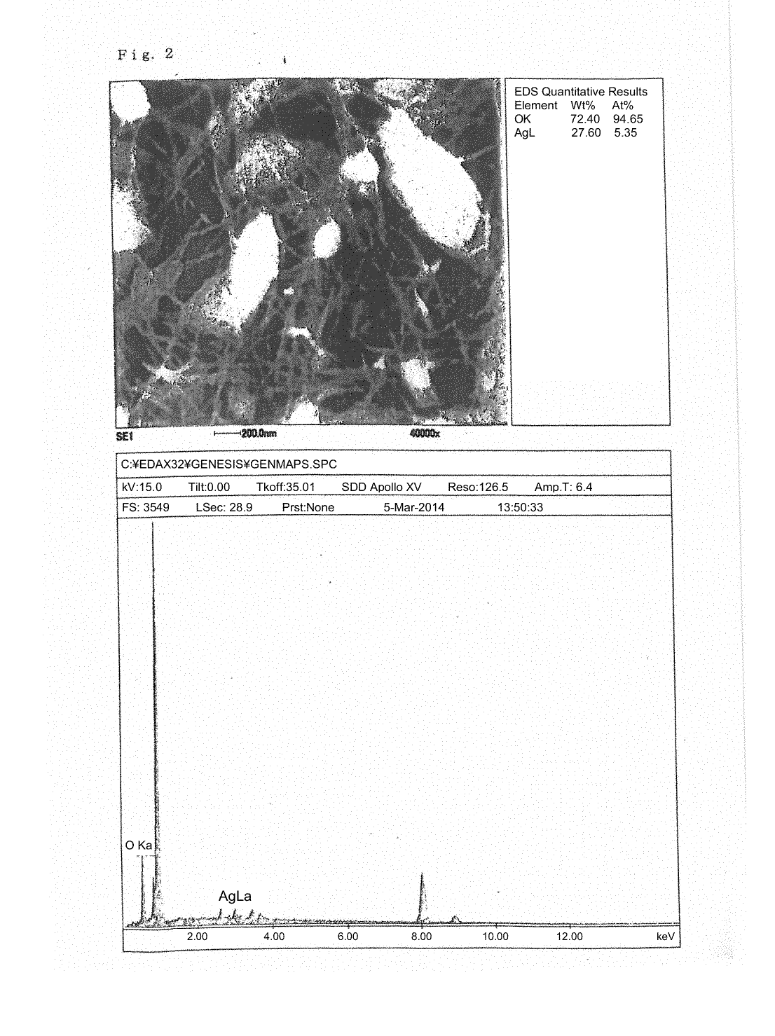

As described below in detail, the present inventor forms the silver complex quantum crystal by allowing the silver thiosulfate aqueous solution to agglomerate on a copper alloy using a chemical reduction method. If such silver complex is alkali treated (sodium hypochlorite treated) in the presence of halogen ion, a superstructure (meso structure) of needle-like nano crystal of silver oxide containing silver peroxide is formed with a help from formation of nucleus of silver chloride by the chemical reaction as mentioned below (FIG. 2).

Na₂S₂O₃+4NaClO+H₂O→Na₂SO₄+H₂SO₄+4NaCl

Ag⁺+NaCl→AgCl+Na⁺

Ag⁺+3NaOCl→2AgCl+NaClO₃+2Na⁺

Ag⁺+OH⁻→AgOH

2Ag⁺+2OH→Ag₂O+H₂O

This needle-like nano crystal has a negative charge in water while a free DNA resulting from undifferentiated cell in a sample has a positive charge due to the fact that the DNA twines around a histone. Thus, the protein resulting from the undifferentiated cell has a positive charge and thus selectively be absorbed on the needle-like nano structure. In addition, the needle-like nano crystal group of silver halide or composite of silver oxide containing halogen is changed into silver metal nanoparticles by a reductive reaction due to a laser irradiation. Thus, a surface plasmon enhancing effect occurs by the laser irradiation. As a result, a chromatin peak occurs in the surface-enhanced Raman scattering (SERS) spectrum for detecting the undifferentiated cell as typified by the absorbed free DNA.

The present invention will be described by way of Examples. However, the present invention should not be construed as being limited by the Examples. Next will be specifically described the embodiments of the present invention by reference to figures below.

(Example of Manufacturing Protein Absorption Chip)

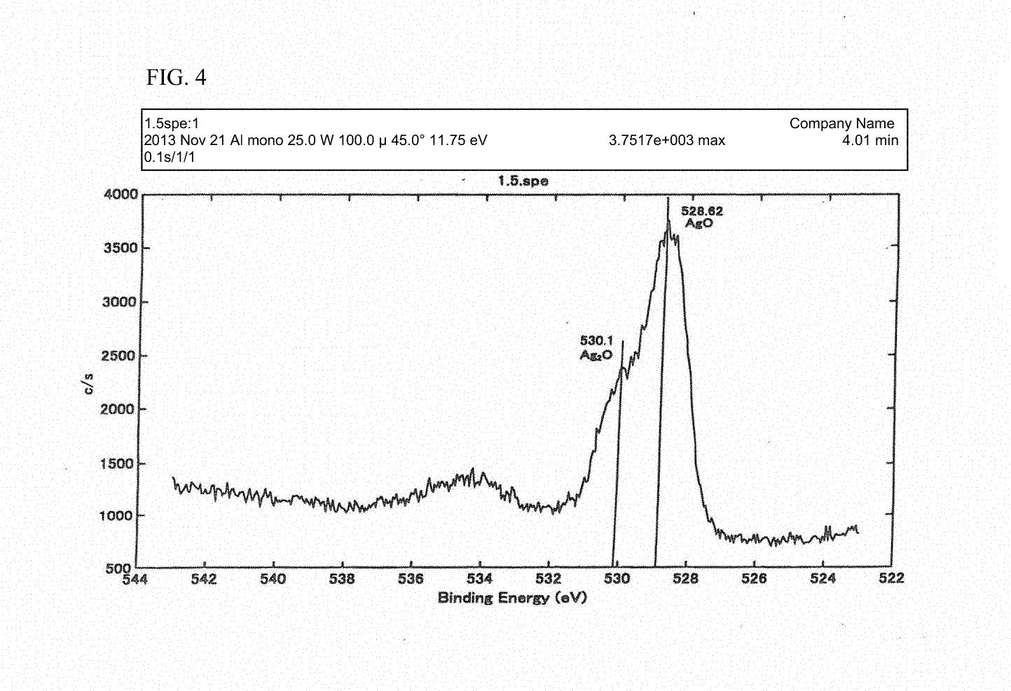

As shown in FIG. 4, 1000 ppm of silver thiosulfate aqueous solution was prepared, and one drop of the aqueous solution was put onto a phosphor-bronze plate. After 3 minutes the drop was blown away, the quantum crystals were obtained as shown in SEM image in the right side. The SEM image shows that the resulting crystal is a thin hexagonal cylinder-like crystal having 100 nm of thickness, wherein asperities of several nano order were formed on the surface. A facet characteristic of metal nano crystal was not found. First, a hexagonal quantum crystal is formed, and the crystals are growing with keeping its shape. From EDS spectrum (elemental analysis), it is found that elements originating from the silver or the complex ligand were detected.

(Consideration of Preparation of the Quantum Crystal)

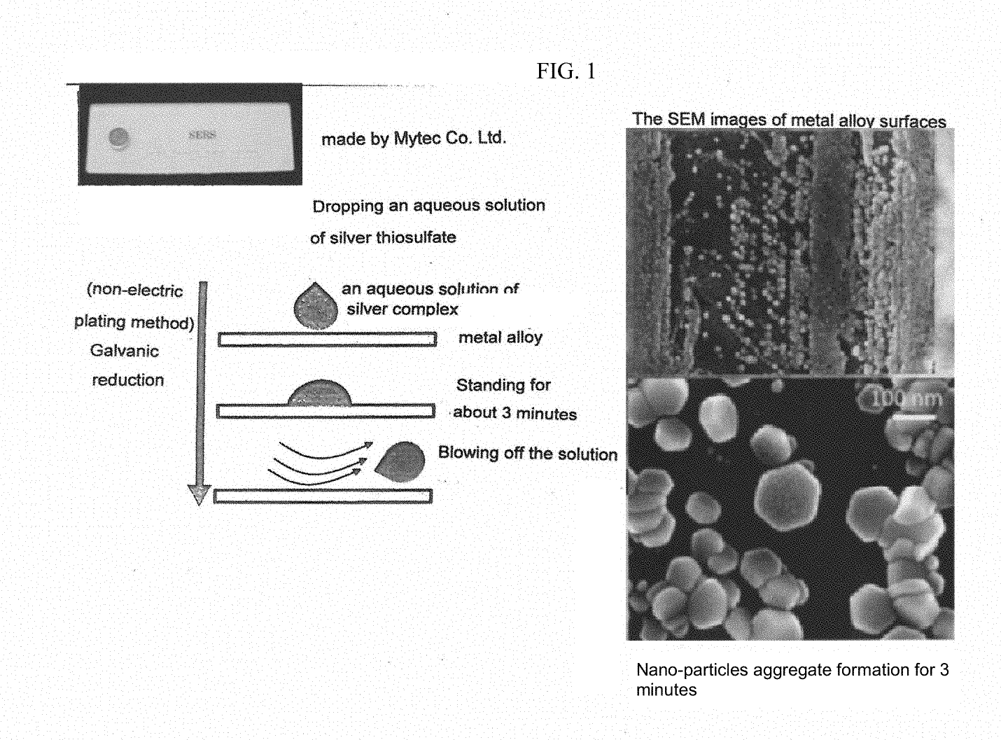

It was confirmed from the SEM image (FIG. 1) that in case of 1000 ppm of silver thiosulfate complex aqueous solution, the quantum crystals were formed as crystals like hexagonal cylinder having about 100 nm of diameter, and that each of the hexagonal cylinder-like quantum crystals has asperities of several nanometer order. However, a facet characteristic of metal nano crystal was not found. In light of detection of EDS elemental analysis that the elements originating from the silver or the complex ligand were found, it is assumed that 1) the resulting quantum crystals are entirety made of the silver complex nano crystal, and 2) the asperities formed on its surface shows formation of the quantum dots from clusters due to expansion of the silver contained in the complex. In the present invention, the followings would be important for forming the quantum crystals; 1) the complex aqueous solution would be in the dilute range of 500-2000 ppm, 2) an electrode potential of supported metal would be slightly base, that is, not noble with respect to an equilibrium potential of the metal complex aqueous solution, 3) the metal complex would agglomerate by the difference in electrode potential. 1000 ppm of silver thiourea complex aqueous solution could show the same function as that of silver thiosulfate solution.

(Consideration of the Meso Crystal of Silver Oxide: No. 1)

When drops of 5% of sodium hypochlorite aqueous solution were put onto the quantum crystal substrate to treat the substrate for two minutes and subsequently the drops were removed. The crystal structure was observed as shown in FIG. 2. As seen in FIG. 2, the needle-like crystals, the rugby ball-like clusters and the large cluster were found. From analyzation by EDS spectrum (element analysis), any chlorine was not observed, and the silver and the oxygen was dominantly found in FIG. 2, although both of the needle-like crystals would be formed of the composite crystal of silver chloride and silver oxide, from the following reaction.

Na₂S₂O₃+4NaClO+H₂O→Na₂SO₄+H₂SO₄+4NaCl (1)

Ag⁺+NaCl→AgCl+Na⁺ (2)

Ag⁺+3NaOCl→2AgCl+NaClO₃+2Na⁺ (3)

Ag⁺+OH^{-AgOH} (4)

2Ag⁺+2OH→Ag₂O+H₂O tm (5)

Thus, it is thought that the meso crystal according to the present invention can be formed from the silver ion and the thiosulfate ion by an alkali oxidation reaction in the presence of chlorine ion. It is normal that the silver oxide is only formed in the normal aqueous solution. However, it is surprising that the silver peroxide is dominantly formed from the XPS measurements mentioned below.

### (Consideration of the Meso Crystal of Silver Oxide: No.2)

XPS Measurements:

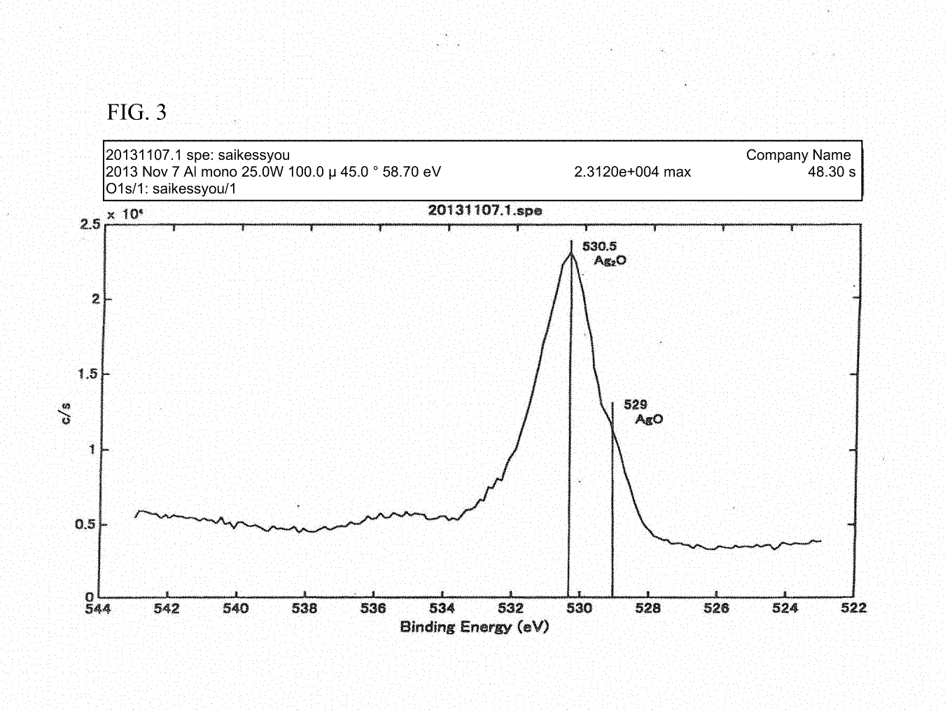

25 μl of sodium hypochlorite aqueous solution was given onto the quantum crystal substrate for two minutes to form the meso crystal recrystallized on the substrate. The substrate was analyzed as to Ag and O by XPS measurement without etching (Type of used equipment: ULVAC-PHI, Inc./PH15000 VersaProbe II (scanning X-ray photoelectron spectrometer)). In comparison, Ag contained in powder of silver oxide and Ag contained in powder of silver chloride were analyzed. On the other hand, the recrystallization substrate is etched by argon gas cluster ion gun for 5 minutes and subjected to the XPS measurement as to Ag and O. The XPS measurement of FIGS. 3 and 4 were analized based on the result of the EDS of FIG. 2, wherein the peak observed at around 529 eV indicates O peak caused by the silver peroxide (AgO) and the peak observed at around 530 eV indicates O peak caused by the silver oxide (Ag₂O). Although the content of oxygen decreases by etching, the O peak caused by the silver peroxide (AgO) at around 529 eV keeps larger than that of the O peak caused by the silver oxide (Ag₂O) at 530 eV, which means that the silver peroxide may be formed near the substrate. We assumed that the catalyst action and the electrode potential of the substrate would cause the above phenomenon, during formation of the meso crystal. In the meanwhile, the EDS measurement with respect to the recrystallization substrate was carried out by JAPAN ELECTRON OPTICS LABORATORY CO., LTD/JSM-7001F (Electron field emission analysis electron scanning microscope) (type of used equipment).

### INDUSTRIAL APPLICABILITY

According to the present invention, it is quite important that the meso crystal can be obtained in the field of the silver oxide. The larger activity of the meso crystal obviously realizes the application of the silver oxide to be expanded and increased. In addition, the performance of silver oxide can be fully improved by preventing the metal oxide nanoparticles from agglomeration and segregation, whereby limitation of the performance could be omitted away.

What is claimed is:

1. A method for preparing a meso crystal of silver oxide containing silver peroxide, comprising;
forming a silver thiosulfate complex crystal on a substrate or a particle made of copper metal or copper alloy, and
treating the substrate or the particle made of copper metal or copper alloy by an alkaline aqueous solution containing halogen ion to obtain a meso crystal of silver oxide containing the silver peroxide.

2. The method for preparing a meso crystal of silver oxide according to claim 1, wherein the silver thiosulfate complex crystal is a quantum crystal of metal complex formed on the substrate or the particle made of copper metal or copper alloy,
while the alkaline aqueous solution containing halogen ion is a sodium hypochlorite aqueous solution, and
wherein a meso structure is a superstructure of silver oxide containing silver peroxide, made by self-organization and three-dimensionally arrangement in the shape of neuron.

3. A meso crystal of silver oxide having nanometer scale, containing a silver peroxide, the silver oxide nanocrystal being a superstructure three-dimensionally arranged in the shape of a neuron.

4. The meso crystal of silver oxide according to claim 3 provided with properties being negatively charged in water.

5. The meso crystal of silver oxide according to claim 3 provided with properties able to be reduced to a silver nanoparticle by a laser radiation.

6. The meso crystal of silver oxide according to claim 4 provided with properties able to be reduced to a silver nanoparticle by a laser radiation.
